SEQUENCE LISTING (3 sequences)

SEQ ID NO: 1 - protein, 408 aa, Homo sapiens
ATGGCTTGTGGTCTGGTCGCCAGCAACCTGAATCTCAAACCTGGAGAGTGCCTTCGAGTGCGAGGCGAGGTGGCTCCTGACGCTAAGAGCTTCGTGCTGAACCTGGGCAAAGACAGCAACAACCTGTGCCTGCACTTCAACCCTCGCTTCAACGCCCACGGCGACGCCAACACCATCGTGTGCAACAGCAAGGACGGCGGGGCCTGGGGGACCGAGCAGCGGGAGGCTGTCTTTCCCTTCCAGCCTGGAAGTGTTGCAGAGGTGTGCATCACCTTCGACCAGGCCAACCTGACCGTCAAGCTGCCAGATGGATACGAATTCAAGTTCCCCAACCGCCTCAACCTGGAGGCCATCAACTACATGGCAGCTGACGGTGACTTCAAGATCAAATGTGTGGCCTTTGACTGA

SEQ ID NO: 2 - protein, 482 aa, Artificial Sequence
  Recombinany galectin-1 rHsGal-1
GTTTAACTTTAAGAAGGAGATATACACATGGCTTGTGGTCTGGTCGCCAGCAACCTGAATCTCAAACCTGGAGAGTGCCTTCGAGTGCGAGGCGAGGTGGCTCCTGACGCTAAGAGCTTCGTGCTGAACCTGGGCAAAGACAGCAACAACCTGTGCCTGCACTTCAACCCTCGCTTCAACGCCCACGGCGACGCCAACACCATCGTGTGCAACAGCAAGGACGGCGGGGCCTGGGGGACCGAGCAGCGGGAGGCTGTCTTTCCCTTCCAGCCTGGAAGTGTTGCAGAGGTGTGCATCACCTTCGACCAGGCCAACCTGACCGTCAAGCTGCCAGATGGATACGAATTCAAGTTCCCCAACCGCCTCAACCTGGAGGCCATCAACTACATGGCAGCTGACGGTGACTTCAAGATCAAATGTGTGGCCTTTGACGGCTCCCATCACCATCACCATCACTGACATATGAAAGAAACCGCTGCTGC

SEQ ID NO: 3 - protein, 831 aa, Artificial Sequence
  Galectin-1 fixed dimer
CTCGAGATGGCTTGCGGCTTGGTAGCTAGTAATCTTAACTTGAAACCCGGAGAGTGTCTGCGAGTGAGAGGAGAGGTGGCTCCCGACGCCAAGAGTTTCGTGCTCAACCTGGGAAAAGACTCCAATAACTTGTGTCTCCACTTCAATCCTAGGTTTAATGCCCATGGGGACGCTAACACAATCGTGTGCAACAGCAAGGATGGGGGCGCCTGGGGCACCGAGCAGCGAGAGGCCGTTTTCCCGTTCCAACCCGGGTCCGTAGCTGAGGTTTGCATCACCTTCGACCAGGCCAACCTGACAGTGAAGCTCCCCGATGGATATGAGTTTAAATTCCCTAATCGTCTGAATCTGGAAGCCATTAACTACATGGCCGCCGACGGTGACTTTAAAATAAAGTGCGTGGCATTCGACGGAGGCATGGCTTGCGGCCTCGTTGCCTCCAACCTCAATCTTAAGCCAGGTGAGTGCCTGCGCGTAAGAGGCGAGGTCGCCCCCGACGCTAAGTCATTCGTGCTCAATTTGGGCAAAGATTCAAATAATCTGTGCCTTCACTTCAACCCCCGGTTTAACGCTCACGGCGACGCTAATACTATAGTGTGTAACTCTAAAGACGGAGGAGCCTGGGGCACTGAACAGAGAGAGGCGGTCTTTCCTTTTCAGCCTGGCTCAGTTGCCGAGGTGTGTATCACTTTTGATCAGGCAAACCTCACCGTGAAACTCCCCGATGGATACGAGTTTAAATTCCCGAACCGCCTGAACCTGGAAGCTATTAACTATATGGCGGCAGACGGAGATTTTAAAATCAAATGTGTTGCTTTCGACTAGCATATG

(19) United States
(12) Patent Application Publication
(10) Pub. No.: US 2023/0165937 A1
(43) Pub. Date: Jun. 1, 2023

(54) GALECTIN-1 IMMUNOMODULATION AND MYOGENIC IMPROVEMENTS IN MUSCLE DISEASES AND AUTOIMMUINE DISORDERS

(71) Applicants: Pam VAN RY, Provo, UT (US); Mary VALLECILLO, Provo, UT (US); BRIGHAM YOUNG UNIVERSITY, Provo, UT (US)

(72) Inventors: Pam VAN RY, Provo, UT (US); Mary VALLECILLO, Provo, UT (US)

(21) Appl. No.: 17/995,794

(22) PCT Filed: Apr. 7, 2021
(86) PCT No.: PCT/US2021/026232

§ 371 Date: Oct. 7, 2022

Related U.S. Application Data
(60) Provisional application No. 63/006,575, filed on Apr. 7, 2020.

(51) Int. Cl. A61K 38/17; A61P 21/00

(52) U.S. Cl. CPC A61K 38/1732; A61P 21/00

(57) ABSTRACT

Limb-girdle muscular dystrophy type 2B (LGMD2B) is caused by mutations in the dysferlin gene, resulting in non-functional dysferlin, a key protein found in muscle membrane. Treatment options available for patients are chiefly palliative in nature and focus on maintaining ambulation. A method of treating LGMD2B is disclosed herein. The method includes administering to a patient a suitable amount of a galectin protein or fragment thereof. Treatment with a recombinant galectin promoted myogenic maturation as indicated through improvements in size, myotube alignment, myoblast migration, and membrane repair capacity in dysferlin-deficient myo tubes, explant myofibers and mice.

### REFERENCE TO RELATED APPLICATIONS

This application claims the benefit under 35 U.S.C. 119(e) of U.S. Provisional Patent Application No. 63/006,575, filed Apr. 7, 2020 and of U.S. Provisional Patent Application No. 63/161,027, filed Mar. 15, 2021. The disclosure of these priority applications are incorporated herein in their entirety.

### REFERENCE TO SEQUENCE LISTING

A sequence listing entitled “Galectin-1_ST25.txt” is an ASCII text file and is incorporated herein by reference in its entirety. The text file was created on Apr. 5, 2021 and is 14.9 KB in size.

### BACKGROUND

### 1. Field of the Invention

Muscular dystrophies include a heterogeneous group of over thirty different muscle diseases that weaken skeletal muscle. The most common muscular dystrophy is Duchenne muscular dystrophy while limb-girdle muscular dystrophies are rare. Limb-girdle muscular dystrophies are a heterogeneous group of genetic disorders included many distinct sub-types.

Muscular dystrophies are notoriously difficult to treat and there is no cure for the disease. At least one reason contributing to the challenge of treating muscular dystrophies is their heterogeneity and diversity. For example, Duchenne muscular dystrophy is characterized by in part by dystrophin-deficient individuals, while Limb-girdle muscular dystrophy 2B (LGMD2B) is characterized in part by dysferlin-deficient individuals. For at least this reason, treatments for Duchenne muscular dystrophy are not expected to treat LGMD2B.

Several therapies that were reported to reduce severity of disease symptoms in dystrophin-deficient mice failed to ameliorate symptoms in dysferlin-deficient mice. In some cases, treatments that are successful for one type of muscular dystrophy adversely affect outcomes in other types of muscular dystrophies. Ibuprofen is known to alleviate severity of muscular dystrophies associated with dystrophin deficiency in mice. When ibuprofen was used to treat dysferlin-deficient mice, however, no effect was seen on muscle morphology and treadmill running was reduced by 40% (see Collier, et al., Journal of Pharmacology and Experimental Therapeutics, 2018, 364:4, p. 409-419). In another study, the steroidal drug, deflazacort, failed in a double-blind, placebo-controlled clinical trial for treating LGMD2B (see, Walter et al., Orphanet Journal of Rare Diseases, 2013, 8:26). Even though steroids have documented efficacy against Duchenne muscular dystrophy, deflazacort failed in treating patients with LGMD2B.

LGMD2B belongs to a family of muscular dystrophies called dysferlinopathies. The incidence of this disease ranges from 1:1,300 to 1:200,000, with certain geographic locations and ethnic populations more heavily impacted than others. Patients with this disease present muscle degeneration and weakness beginning in the second decade of life and often exhibit complete loss of ambulation by the third decade of life.

Symptoms of LGMD2B stem from mutations in the DYSF gene, which encodes for the dysferlin protein. Dysferlin is a 230 kDa transmembrane protein heavily involved in Ca²⁺ signaling in adult myocytes. Mutations to the dysferlin protein lead to aberrant Ca²⁺ signaling, causing poor membrane repair, myogenesis, and muscle degeneration. Dysferlin-deficient myoblasts show decreased myogenesis, but the direct influence of dysferlin on this process is unclear. Membrane repair is a complex process involving multiple pathways with the purpose of restoring compromised membrane integrity.

Current drug treatments for LGMD2B are limited and focus on mitigating the effects of chronic inflammation. Other palliative treatment options include muscle strengthening and patient education regarding preventative measures to reduce muscle injury. There is an unmet need in the field for viable, long-term therapeutic options. Glucocorticoid treatments have been used to modulate impaired membrane stability and inflammatory response in many muscular dystrophies. Regular glucocorticoid treatment, however, has marginal or detrimental effects in patients with LGMD2B. Therefore, due to the lack of current viable treatments, a therapeutic that can increase myogenic potential and membrane repair would be most beneficial to patients and their families.

### BRIEF SUMMARY

A method of treating limb-girdle muscular dystrophy type 2B in a patient is provided. The method includes administering a suitable amount of a galectin protein, a fragment thereof, or a synthetic variant thereof.

In some aspects, the galectin protein is SEQ ID NO: 1 or SEQ ID NO: 2.

In some aspects, the galectin protein is a fixed dimer of galectin-1.

A method of treating limb-girdle muscular dystrophy type 2B in a patient is provided. The method includes administering about 0.2 mg/kg to about 20 mg/kg of a galectin protein, a fragment thereof, or synthetic variant thereof, wherein the administration is via intraperitoneal injection.

The foregoing broadly outlines the features and technical advantages of the present disclosure in order that the detailed description that follows may be better understood. Additional features and advantages of the disclosure will be described hereinafter that form the subject of the claims of this application. It will be appreciated by those of skill in the art that the conception and specific aspects disclosed herein may be readily utilized as a basis for modifying or designing other aspects for carrying out the same purposes of the present disclosure within the spirit and scope of the disclosure and provided in the appended claims.

### BRIEF DESCRIPTION OF THE SEVERAL VIEWS OF THE DRAWINGS

A detailed description of the invention is hereafter provided with specific reference being made to the drawings in which:

FIG. 1A shows Quantification of myogenin after 72-hour treatment with varying concentrations of rHsGal-1.

FIG. 1B shows western blot images of myogenin at different rHsGal-1 treatments.

FIGS. 1C-1F show quantification of myogenic markers MHC (FIG. 1C), Pax7 (FIG. 1D), MyoD (FIG. 1E), and Myf5 (FIG. 1F) in A/J^{-/-} myotubes after 72-hour treatment with 0.11 μM rHsGal-1.

FIGS. 1G-1H show quantification of Gal-1 (FIG. 1G) and His.H8 (FIG. 1H) in A/J^{-/-} myotubes after 72-hour treatment with 0.11 μM rHsGal-1.

FIG. 1I shows western blot images of myogenic markers (Pax7, Myf5, MyoD, and MHC) and of mouse Gal-1 and His Tagged rHsGal-1.

FIG. 1J shows RT-qPCR quantification of LGALS1 transcript between A/J WT, A/J^{-/-} NT, and A/J^{-/-} 0.11 μM rHsGal-1 treated myotubes.

FIG. 2A shows representative images of A/J cells cultured and immunostained with Phalloidin and DAPI. FIG. 2B. Representative images of A/J cells cultured and immunostained with MHC (red) and DAPI. FIG. 2C. Representative images of A/J cells cultured and immunostained with Myf5, Phalloidin, and DAPI.

FIG. 2D shows average number of nuclei per myotube compared between WT (n=1608 nuclei, 187 myotubes, 10 fields), NT (n=1587 nuclei, 215 myotubes, 9 fields), and 0.11 μM rHsGal-1 treated (n=2476 nuclei, 166 myotubes, 13 fields) groups.

FIG. 2E shows fusion index between WT, NT, and 0.11 μM rHsGal-1 treated myotube groups.

FIG. 3A shows representative images of FM 1-43 dye accumulation in NT and 48 hours 0.11 μM rHsGal-1 treated A/J^{-/-} myotubes after injury with UV laser. White arrows indicate site of injury.

FIG. 3B shows quantification of the change in fluorescent intensity inside A/J^{-/-} myotubes following laser injury when treated with 0.11 μM rHsGal-1 for 10 min and 48 h compared to WT A/J^{+/+} and NT A/J^{-/-} myotubes.

FIG. 3C shows change in the fluorescent intensity in 0.11 μM rHsGal-1 treated A/J myotubes supplemented with lactose and sucrose compared to NT A/J^{-/-} myotubes.

FIG. 3D shows change in the fluoresce intensity in 0.11 μM rHsGal-1 treated A/J^{+/+} myotubes supplemented with or without EGTA and rHsGal-1 compared to WT A/J^{+/+} and NT A/J^{-/-} myotubes.

FIG. 4A shows representative images at time points 0s, 30s, 60s, 90s of FM1-43 dye accumulation in Bla/J mouse fibers upon laser injury with a 405 nm laser. White arrows indicate site of injury.

FIG. 4B shows quantification of the total change of fluorescence in Bla/J myofibers.

FIG. 4C shows quantification of the total change of fluorescence in Dysf^{-/-} myofibers post injury.

FIG. 4D shows quantification of the change in fluorescence in C57BL/6-WT myofibers after treatment with or without rHsGal-1.

FIG. 4E shows quantification of the change in fluorescence in C57BL/6-WT myofibers after treatment with or without EGTA and rHsGal-1.

FIGS. 5A-5C show quantification of the total change of fluorescence in Bla/J myofibers treated with various doses of rHsGal-1.

FIG. 6 shows quantification of the total change of fluorescence in Bla/J myofibers treated with a dose of 2.7 mg/kg of rHsGal-1 twice per week.

FIG. 7 shows quantification of the total change of fluorescence in Bla/J myofibers treated with a dose of 2.7 mg/kg of rHsGal-1 once per week for one month.

FIG. 8 shows quantification of the total change of fluorescence in Bla/J myofibers treated with a dose of 0.27 mg/kg of rHsGal-1 once per week for one month.

### DETAILED DESCRIPTION

Various aspects are described below with reference to the drawings. The relationship and functioning of the various elements of the aspects may better be understood by reference to the following detailed description. However, aspects are not limited to those illustrated in the drawings or explicitly described below. It should be understood that the drawings are not necessarily to scale, and in certain instances details may have been omitted that are not necessary for an understanding of aspects disclosed herein, such as conventional fabrication and assembly. Headings are provided for the convenience of the reader and to assist organization of the disclosure and should not be construed to limit or otherwise define the scope of the invention.

A method of treating LGMD2B in a patient is provided herein. The method includes administering a suitable amount of a galectin protein, a fragment thereof, or synthetic variant thereof.

The manner in which the galectin protein is administered is not particularly limited. In some aspects, the protein is administered via intraperitoneal injection or via intravenous injection.

The suitable amount of the galectin protein, fragment thereof, or synthetic variant is from about 0.2 mg/kg to about 20 mg/kg administered via intraperitoneal injection. In some aspects, the suitable amount administered via intraperitoneal injection is from about 0.2 mg/kg to about 15 mg/kg, from about 0.2 mg/kg to about 10 mg/kg, from about 0.5 mg/kg to about 10 mg/kg, from about 1 mg/kg to about 10 mg/kg, from about 1 mg/kg to about 8 mg/kg, from about 2 mg/kg to about 4 mg/kg, or from about 1 mg/kg to about 5 mg/kg. In some aspects, the suitable amount administered via intraperitoneal injection is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg. In some aspects, the suitable amount administered via intraperitoneal injection is about 2.7 mg/kg.

The suitable amount of the galectin protein, fragment thereof, or synthetic variant is from about 0.01 mg/kg to about 5 mg/kg administered intravenously. In some aspects, the suitable amount administered via intravenous injection is about 0.05 mg/kg, about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, or about 4.5 mg/kg.

The dosing frequency can be daily, weekly, or every other day. In some aspects, the galectin protein, fragment thereof, or synthetic variant is dosed daily. In some aspects, the galectin protein, fragment thereof, or synthetic variant is dosed every other day. In some aspects, the galectin protein, fragment thereof, or synthetic variant is dosed weekly.

As used herein, the term “fragment” refers to any peptide containing a portion of the galectin-1 protein amino acid sequence (SEQ ID NO: 1).

As used herein, the term “synthetic variant” refers to proteins having at least about 90% identity with SEQ ID NO: 1.

In some aspects, the galectin protein is a recombinant galectin-1 protein. The recombinant galectin-1 protein can be SEQ ID NO: 1, SEQ ID NO: 2, or SEQ ID NO: 3. In some aspects, the galectin protein is SEQ ID NO: 1. In some aspects, the galectin protein is SEQ ID NO: 2. In some aspects, the galectin protein is SEQ ID NO: 3.

The method described herein can further include decreasing muscle damage in the patient, increasing muscle repair in the patient, or increasing muscle function in the patient.

In some aspects, the galectin protein is a fixed galectin-1 mimetics such as a dimer, trimer, or tetramer. In some aspects, the galectin protein is a fixed dimer. An example of a fixed dimer is SEQ ID NO: 3.

In some aspects, the galectin protein can be formulated with lipids. Examples of lipid formulations include, but are not limited to, liposomes, micelles, lipid cochleates, and lipid microtubules.

In some aspects, the galectin protein can be encapsultated in poly(D,L-lactic-coglycolic-acid) (PLGA) microspheres.

In some aspects, the galectin protein can be modified with a synthetic polymer such as polyethylene glycol (PEG), polyglutamic acid, or, hydroxyethyl starch. In some aspects, the galectin protein can be hyperglycosylated by the attachment of additional carbohydrates to the protein.

Galectin-1 (Gal-1; SEQ ID NO: 1) is a small, non-glycosylated protein encoded by the LGALS1 gene with a carbohydrate recognition domain (CRD) that is highly conserved between all mammals with an 88% homology. Mouse and human Gal-1 have minor structural differences, but the carbohydrate recognition residues are 100% conserved. Mice lacking Gal-1 showed a reduction in myoblast fusion and muscle regeneration. Recombinant human galectin-1 (rHsGal-1) has shown efficacy in reducing disease pathologies in murine models of Duchenne Muscular Dystrophy (DMD) through stabilization of the sarcolemma. Treatments for DMD, however, are not predictably effective for LGMD2B.

We explore the effects of rHsGal-1 treatment in A/J dysferlin-deficient (A/J^{-/-}) cells and ex-vivo muscle assessment using Dysf^{-/-} (B6.129.Dysftm1Kcam/J), Bla/J (B6.A-Dysfprmd/GeneJ), and BL/6 (C57BL/6) mice. This study shows that Gal-1 treatment increases myogenic transcription factors leading to enhanced myotube formation in A/J^{-/-} myotubes and increased membrane repair capacity in A/J^{-/-} myotubes as well as Dysf^{-/-} and WT and dysferlin-deficient myofibers. Additionally, this work reveals that the carbohydrate recognition domain (CRD) of Gal-1 is necessary for improved repair capacity and that the impact of Gal-1 on membrane repair is Ca²⁺-independent in both diseased and non-diseased models. Together, these findings support Gal-1 therapeutic applications for LGMD2B.

The following examples provide and illustrate certain features and/or aspects of the disclosure. The examples should not be construed to limit the disclosure to the particular features or aspects described therein.

### EXAMPLES

### Example 1

Recombinant Human Galectin-1 (rHsGal-1) Production and Purification

The human Galectin-1 gblock LGALS1 gene fragments were produced as doubled-stranded DNA using high fidelity polymerase. The LGALS1 gblock was cloned into the pET29b (+) vector using NEBuilder® HiFi DNA Assembly Cloning Kit. The product was purified following the E.Z.N.A.® Plasmid DNA Mini Kit I protocol and the DNA sequence was confirmed by Eton-Bioscience, Inc. The cloned vector was transformed into BL21(DE3) competent E. coli cells (High Efficiency, NEB #C2527H) grown and induced with 0.1 mM IPTG. rHsGal-1 was purified using the Cobalt Talon Metal Affinity Resin protocol in a poly-Prep® Chromatography column and imidazole elution buffer. Purified rHsGal-1 was then filtered and dialyzed three times for a total of 24 hours in PBS at 4° C. Endotoxin levels were measured using LAL Chromogenic Endotoxin Quantitation Kit. All endotoxin levels of purified rHsGal-1 were below the FDA limit of 0.5 EU/ml at >0.1 EU/ml. Purified rHsGal-1 was conjugated with Alexa Fluor 647 following the protocol provided with the protein labeling kit. The concentration of both rHsGAL-1 and Alexa Fluor 647 labeled rHsGal-1 was determined with the Pierce™ BCA Protein Assay Kit.

Gal-1 induces skeletal muscle differentiation and decreases disease manifestation in DMD. Exogenous Gal-1 may positively modulate different pathologies in LGMD2B. To explore the effects of Gal-1 treatment in Dysf-deficient models, endotoxin-free rHsGal-1 was produced using the pET29b(+) vector with a C-terminal 6×Histidine tag for easy detection during purification and expression steps. Purification and detection analyses were made by total protein stains and western blot.

### Cell Culture

Immortalized murine myoblasts H2K A/J^{-/-}, [A/J^{-/-}], and H2K A/J WT, [WT], were cultured. Myoblasts were then plated onto glass-bottomed, collagen coated dishes sterilized with gamma-irradiation, seeded at a density of 5,555 cells/cm² and incubated at 33° C. in 10% CO². Myotubes were obtained from confluent myoblasts after 2 to 4 days in differentiation media supplemented with or without rHsGal-1 (0.014 μM-0.22 μM). Differentiation media and treatments were changed every other day.

### Western Blotting

Myotubes (at 2 to 4 days) were obtained as described above. Whole cell lysates were prepared using RIPA lysis buffer (10 mM Tris-Cl (pH 8.0), 1 mM EDTA, 1% Triton X-100, 0.1% sodium deoxycholate, 0.1% SDS, 140 mM NaCl, and 1 mM PMSF) and Halt™ Protease and Phosphatase Inhibitor Single-Use Cocktail (100×). Protein concentration was determined using the Pierce™ BCA Protein Assay Kit. Proteins samples were separated under standard conditions on 6%-20% SDS-PAGE gels and transferred onto Nitrocellulose Membranes 0.2 μm through electro blotting. After blocking with 5% w/v non-fat dry milk in 1× TBST), membranes were probed overnight for the following mouse, rabbit, or goat monoclonal and polyclonal antibodies: 6×-His Tag Monoclonal Antibody (HIS.H8), Galectin-1 Monoclonal Antibody (6C8.4-1) (Cat. No. 43-7400, Invitrogen 1:1000), Myogenin (FD5, DSHB, 0.2 μg/mL, Pax7 (DSHB, 0.2 μg/mL), Myf5, MyoD, MHC, Annexin A6, Annexin A1, β-Tubulin Loading Control, BT7R, GAPDH, and Anti-β-actin. After washing primary antibodies, blots were probed using the following secondary antibodies IRDye® 800CW Donkey Anti-Rabbit IgG (H+L), Goat anti-Mouse IgG (H+L) Highly Cross-Adsorbed Secondary Antibody, Alexa Fluor Plus 800, Goat anti-Mouse IgG (H+L) Highly Cross-Adsorbed Secondary Antibody, Alexa Fluor 680, and IRDye® 680RD Donkey Anti-Goat IgG. The blots were developed using the Odyssey CLx. Quantifications were done by using ImageJ.

### Immunofluorescence

A/J^{-/-} and A/J WT myotubes cultured onto 35 mm Glass Bottom Microwell Dishes were fixed in 4% paraformaldehyde, permeabilized in 0.1% triton X-100 (in PBS), and blocked using MOM IgG blocking solution for 1 h at room temperature. The myotubes were then incubated overnight at 4° C. with the appropriate primary antibody: Alexa Fluor 647/Phalloidin, Myf5, MHC, CellBrite™ Cytoplasmic Membrane Dyes. Nuclei were counterstained with Hoeschst 33342 and 4′,6-diamindino-2-phenylindole (DAPI). Blots were probed using the following secondary antibodies: Fluorescein (FITC) AffiniPure Rabbit Anti-Goat IgG, Fc fragment specific, Goat anti-Mouse IgG (H+L) Highly Cross-Adsorbed Secondary Antibody, Alexa Fluor Plus 488, Goat anti-Mouse IgG (H+L) Highly Cross-Adsorbed Secondary Antibody, Alexa Fluor 680 (Cat. No. A21058, ThermoFisher, 10 μg/ml). Myotubes were mounted on coverslips using ProLong™ Diamond Antifade Mountant (Cat No. P36965, Invitrogen) and dried overnight. Images were taken on the A TCS SP2 two-photon confocal scanning microscope with LASX imaging software (Leica Microsystems Inc., Buffalo Grove, Ill.). 647rHsGal-1 inside-outside fluorescent values were obtained as described in Fitzpatrick et al. Inside-outside ratio was calculated by averaging three ROI from inside a cell and three ROI between cells per image.

### Fusion Index Scoring

A/J^{-/-} and A/J WT myoblasts were plated onto in 35 mm Glass Bottom Microwell Dishes. At 80%-90% confluence, myoblasts were differentiated as described above and were given treatment (0.11 μM rHsGal-1) or not. After three days in differentiation media and treatment, myotubes were fixed, permeabilized, stained and imaged as described above. Fusion index was calculated as the number of nuclei contained within myotubes (cells were considered to be myotubes if they contained three or more nuclei) divided by total number of nuclei. Minimum Feret's Diameter (MFD) was calculated by using ImageJ. Myotubes were outlined using the polygon tool, after which the MFD was calculated with the Feret's Diameter plugin.

### Migration Assay

12-well plates were prepared by placing a silicone insert in the center of each well. A suspension of 145,000 cells/ml (either WT and A/J^{-/-} myoblasts) was prepared in growth media as described above and 70 μl of the suspension was placed into each side of the insert. After 2 days, cells were placed in normal differentiation media or differentiation media supplemented with 0.11 μM rHsGal-1 and incubated for 2 days. To form the wound, the silicone insert was removed 1 h prior to first image after washing with PBS; Rate of migration was calculated over a 48 hour period. Differentiation media or differentiation media supplemented with 0.11 μM rHsGal-1 was then replaced as described above and directly placed into the Incucyte®. Magnification was set to 10× and images were taken every 3 h for 48 h. Images were analyzed with ImageJ.

### Laser Injury Assay

A/J WT and A/J^{-/-} 0.11 μM rHsGal-1 treated or NT myotubes were prepared for laser injury as described above in 35 mm Glass Bottom Microwell Dishes. After washing with PBS, the myotubes were incubated for 10 mM in PBS enriched with or without: 1 mM Ca²⁺ (as CaCl²)), 1 μM intracellular (1,2-Bis(2-aminophenoxy) ethane-N, N, N′,N′-tetraacetic acid tetrakis (acetoxymethyl ester); (BAPTA-AM), DMSO as a vehicle, 1 μM (ethylene glycol-bis(β-aminoethyl ether)-N,N,N′,N′-tetraacetic acid; (EGTA), 20 mM lactose or 20 mM sucrose, and 2.5 μM FM™ 1-43 dye (N-(3-Triethylammoniumpropyl)-4-(4-(Dibutylamino) Styryl) Pyridinium Dibromide)3,5 for 5 mM before injury. A TCS SP2 two-photon confocal scanning microscope (Leica) was used to injure the membrane of a myotube or myofiber and images were taken before and after the injury event. Pre-injury images depict uninjured myofibers. Myoblasts were not used in injury protocols, only cells with greater than 3 nuclei were counted as myotubes. The myotube was injured with a 405 nm UV laser at 100% power on a HCX PL APO CS 63.0×1.40 oil-objective lens. Post-injury images were taken every 5 sec for a total of 150 sec. At least three different myotubes were selected to be injured in each dish. The total change in fluorescence intensity of FM™ 1-43 dye at the site of the wound for each time point relative to the pre-injury fluorescent intensity was measured using ImageJ.

### Muscle Fiber Isolation

A 12-well plate was prepared. After preparation of digestion plate, C57B6 and Dysf−/− (B6.129-Dysftm1 Kcam/J) mice were euthanized. When the mice were sacrificed, hind limbs were removed and the tibialis anterior, flexor digitorum brevis, and/or gastrocnemius were excised. Next, by using a small-bore pipette, the fibers were transferred to in 35 mm Glass Bottom Microwell Dishes and allowed to attach for at least 15 min Fibers were then treated or not with 0.11 μM rHsGal-1 and kept at 37° C. until injury. At least three different myofibers in each dish were selected to be injured. The total change in fluorescence intensity of FM™ 1-43 dye at the site of the wound for each time point relative to the pre-injury fluorescent intensity was measured using ImageJ.

### Quantitative RT-PCR

Total RNA was isolated from 3 days differentiated A/J WT, A/J^{-/-}, and A/J^{-/-} treated with 0.11 μM rHsGal-1 myotubes (n=6 independent clonal lines for each treatment group) using Quick-RNA™ Miniprep kit. Isolated RNA was reverse transcribed using SuperScript™ IV VILO™ following the manufacturer's instructions. Real-time analysis was performed on an Applied Biosystems® QuantStudio® 5 Real-Time PCR System using TaqMan® Fast Advanced Master Mix and TaqMan® Assays. Relative gene expression levels and statistical significance were calculated by normalizing raw Ct values to 18S, and then by using the ΔΔCt method with Applied Biosystems™ Relative Quantitation Analysis Module software.

### Statistical Analysis

Data analysis were completed by using Tukey's multiple comparison test 1-way and 2-way ANOVA, the Student's t test, Welch's, and Bartlett's test through the GraphPad Prism Software version 8.0. For membrane repair analysis, the data are conferred the averaged values for all the myotubes used in the analysis, and the treatment at individual time points. *p<0.05, **p<0.01, ****p<0.0001.

### Results

rHsGal-1 Increases Myogenic Potential in A/J^{-/-} Myotubes

The formation of myotubes is a multi-step process incorporating migration, adhesion, and alignment, followed by formation of extracellular proteins that coordinate cellular stability. Gal-1 expression levels during myoblast growth, differentiation and repair play a key role in forming healthy skeletal muscle. The lack of Gal-1 leads to poor myotube formation and delays in myoblast fusion.

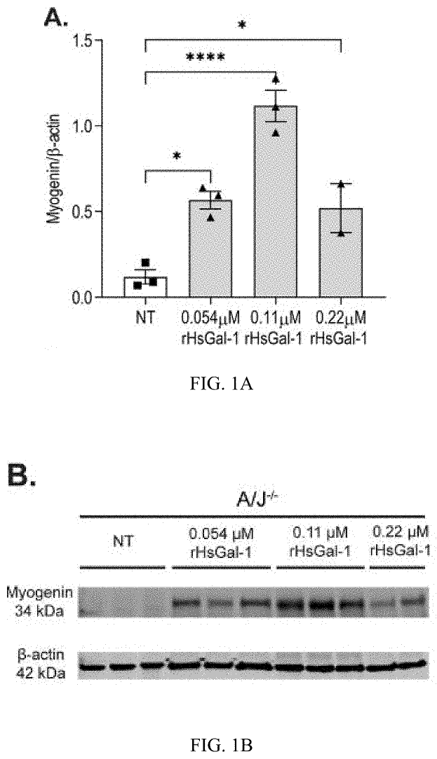

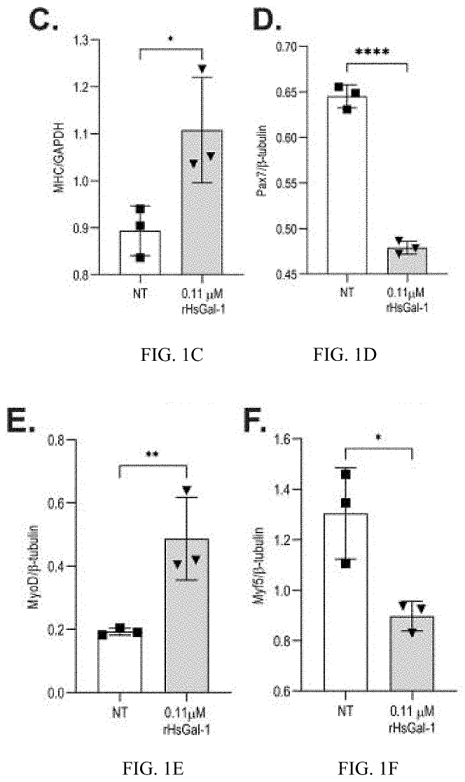

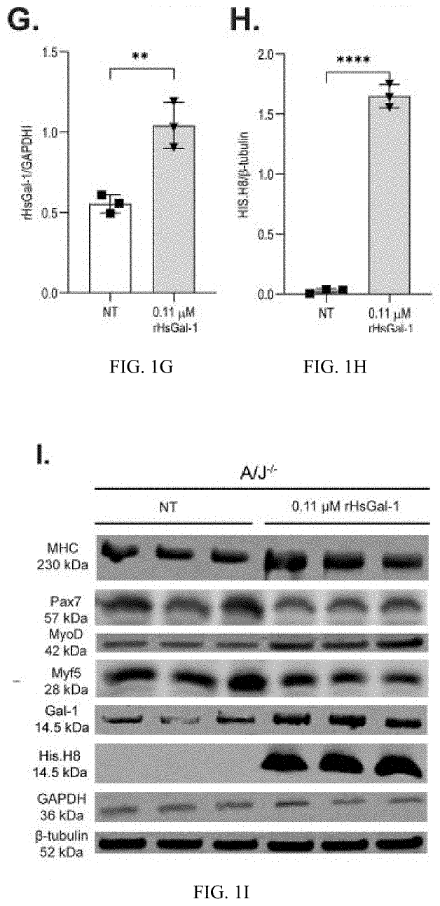

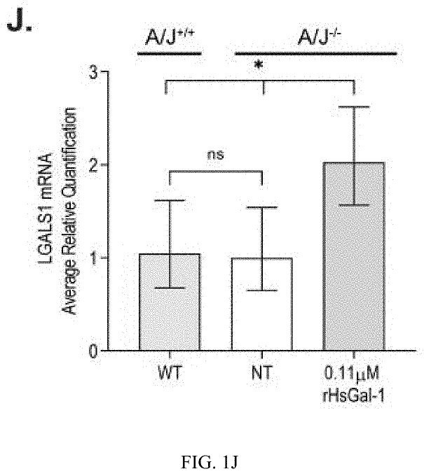

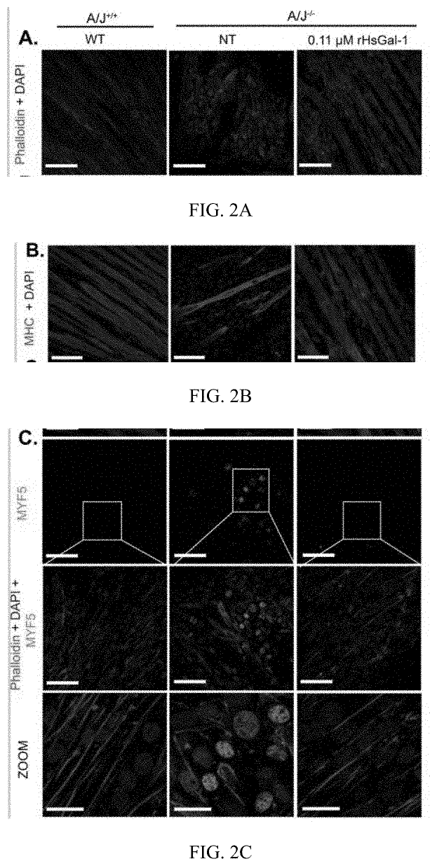

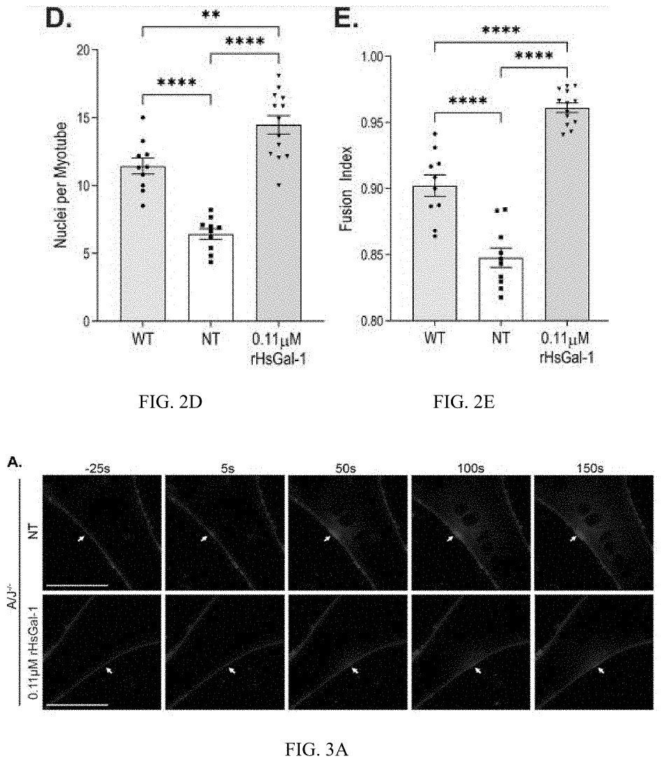

Myogenin is a muscle-specific transcription factor expressed by terminally differentiated myotubes and is known to be decreased in immortalized A/J^{-/-} myotubes. However, after a 72-hour treatment with rHsGal-1, myogenin expression increased in A/J^{-/-} myotubes (FIGS. 1A and 1B). To determine the most efficacious dose of rHsGal-1 required to increase myogenesis, myoblasts either received no treatment (NT) or were treated with three concentrations of rHsGal-1 for 72-hour post differentiation. When compared to NT myoblasts our results show a 4.74-, 9.35- and 4.35-fold increase in myogenin with 0.054 μM, 0.11 μM, and 0.22 μM rHsGal-1 treatment, respectively (FIGS. 1A and 1B). To further investigate changes in myogenic potential, whole cell lysates were examined and levels of early, mid, and late myogenic markers were measured: paired box protein 7 (Pax7), myogenic factor 5 (Myf5), myoblast determination protein (MyoD) and myosin heavy chain (MHC) respectively, after treatment with a dose of 0.11 μM rHsGal-1 (FIG. 1C-1F). Levels of early stage markers decreased, while levels of late stage markers increased by 2.5-fold (MyoD) and 1.46-fold (MHC) when treated with 0.11 μM rHsGal-1 (FIGS. 1C-1F and 1I). The removal of 0.11 μM rHsGal-1 after a 10 min treatment in A/J^{-/-} myotubes followed by 72 hours in differentiation media show no significant difference in Myf5 or MHC expression when compared to NT. The changes in myogenic transcription factors were validated using immunofluorescent imaging. After 48 hour of differentiation, early myotube populations with or without treatment were stained with a nuclear counterstain 4′,6-Diamidino-2-Phenylindole, dihydrochloride (DAPI), an actin filament stain (Phalloidin) and anti-Myf5. Images reveal that there was no Myf5 visible in WT or rHsGal-1 A/J^{-/-} treated myotubes, while Myf5 positive myoblasts are observed in A/J^{-/-} NT (FIG. 2C).

In order to show that rHsGal-1 treatment was the cause, transcript and protein levels of Gal-1 were investigated. RT-qPCR analysis revealed LGALS1 mRNA transcript levels were doubled after a 72 hour 0.11 μM rHsGal-1 treatment post differentiation (FIG. 1J). Increases in rHsGal-1, 6×His-tag protein, and LGALS1 mRNA transcripts levels correlate with rHsGal-1 treatment and suggest a positive feedback loop that ultimately upregulates myogenic transcription factors in diseased cells with a 72-hour treatment (FIG. 1G-1J). The levels of 6×His-tag after 72 hours also indicate that the exogenous Gal-1 is internalized and stable within cell culture.

Gal-1 knockout mice are reported to have decreased myofiber formation. We explored the ability of rHsGal-1 to increase fusion capacity of A/J^{-/-} myotubes by measuring fusion index, alignment and size. Dysferlin-deficient myotubes were stained with Phalloidin or MHC and DAPI in order to determine fusion index (FIGS. 2A and 2B). Treatment with 0.11 μM rHsGal-1 showed a dramatic increase in number of nuclei per myotube (WT=11.4±0.59, NT=6.43±0.37, and rHsGal-1=14.5±0.65) (FIG. 2D) and average fusion index (WT=0.90±0.003, NT=0.85±0.007, rHsGal-1=0.96±0.004) (FIG. 2E). Myotube and myofiber alignment have been shown to lead to improved muscle development and strength. This set of experiments suggest that low doses 48-hour treatment of rHsGal-1 in an in vitro dysferlin-deficient model may increase myogenic potential in myoblasts.

Increased rHsGal-1-Mediated Repair is Dependent on the CRD of rHsGal-1 and Independent of Ca²⁺ in Both Dysferlin-Deficient and Non-Diseased Models

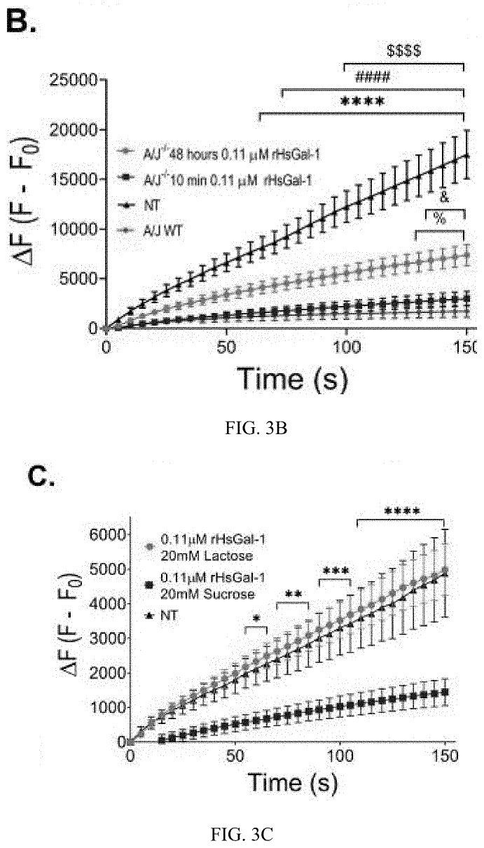

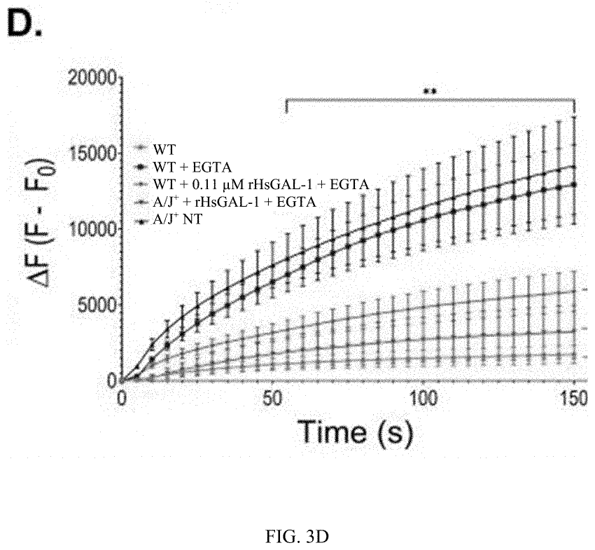

The major pathological feature in LGMD2B is compromised membrane repair. To explore the effectiveness of rHsGal-1 treatment on the membrane repair process, we employed a membrane laser injury assay on dysferlin-deficient myotubes (myotubes defined as having >3 nuclei) in the presence of FM1-43, a lipophilic dye that fluoresces when bound to lipids. We quantified the change in fluorescence after injury; cells with less dye entry indicate better membrane repair (FIG. 3A). These injuries were performed after 10 minutes and 48 hour treatments to evaluate any time-dependencies. After laser injury, we quantified changes in fluorescent intensity to measure effectiveness and kinetics of membrane repair. At 150 seconds post-injury, A/J−/− myotubes treated with 0.11 μM rHsGal-1 for 48 hours had 58% less dye entry than NT, while a 10-minutes' treatment decreased final fluorescent intensity by 83% from NT. WT had 90.1% less dye entry than NT. In comparing dysferlin-deficient treatments to WT cells, after a 10 minutes and 48-hours treatment A/J^{-/-} cell only allowed 7% and 32% more dye than non-diseased cells (FIG. 3B).

To determine the involvement of the Gal-1 carbohydrate recognition in repair capacity, we performed a laser ablation assay in the presence of lactose or sucrose. The CRD of Gal-1 is known to have a binding affinity for lactose whereas sucrose does not interact with the CRD. When A/J^{-/-} myotubes were incubated with 20 mM sucrose and 0.11 μM rHsGal-1 10 minutes prior to treatment, we observed an increase in membrane repair capacity consistent with previous results. However, when rHsGal-1 CRD interactions were inhibited with lactose, we saw no increase in membrane repair (FIG. 3C). We conclude that the CRD plays a crucial role in the membrane repair mechanism of Gal-1.

Non-diseased models show that dysferlin-mediated repair is dependent on intrinsic Ca²⁺ signaling properties of dysferlin. Therefore, dysferlin-deficient muscle fibers are defective in many Ca²⁺ sensitive processes, including membrane repair. We conducted a group of laser injury assays to determine the role of Ca²⁺ in rHsGal-1 mediated. Dysferlin-deficient myotubes treated with 0.11 μM rHsGal-1 for 48 hour had a final change in fluorescent intensity 57% lower than NT A/J^{-/-} myotubes 150 seconds post injury, independent of the presence of Ca²⁺ in their cell media. To better understand the Ca²⁺ independent therapeutic benefit of Gal-1 in A/J^{-/-} myotubes, we quantified final fluorescent intensity in the presence and absence of extracellular (EGTA) and intracellular (BAPTA-AM) calcium chelators. We saw that rHsGal-1 treatment increases membrane repair and mitigates effects of dysferlin-deficiency in the presence of both intracellular and extracellular calcium chelators. Calcium imaging using Fluo-4AM also revealed no increase in Ca²⁺ accumulation at site of injury in A/J^{-/-} 0.11 μM rHsGal-1 treated and NT myotubes, but did find an increase in Ca²⁺ accumulation at the site of injury in A/J WT myotubes. Next, we wanted to determine the positive impact of rHsGal-1 on membrane repair in the presence of dysferlin through A/J^{+/+} WT myotubes. We used EGTA to inhibit the normal, calcium-dependent function of dysferlin in WT myotubes. Our results showed no significant differences in membrane repair between non-treated A/J^{-/-} and WT myotubes treated with EGTA. Although WT myotubes treated with EGTA showed reduced repair due to lack of extracellular Ca²⁺, WT myotubes treated with 0.11 μM rHsGal-1 plus EGTA showed a significant improvement in membrane repair similar to A/J^{-/-} myotubes treated with 0.11 μM rHsGal-1 (FIG. 3D). Even when deprived of Ca²⁺, WT cells treated with Gal-1 are able to alleviate repair defects due to lack of Ca²⁺.

Ex-Vivo rHsGal-1 Treatment Increases Membrane Repair Capacity in Dysf^{-/-} Myofibers

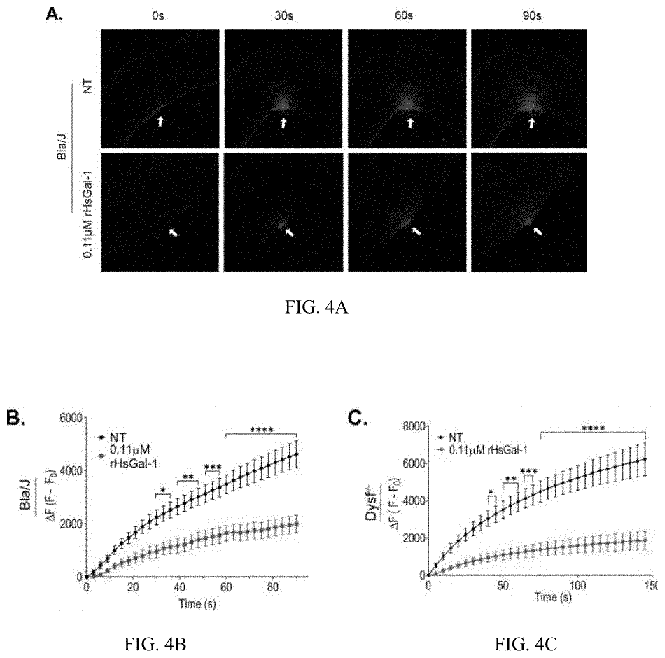

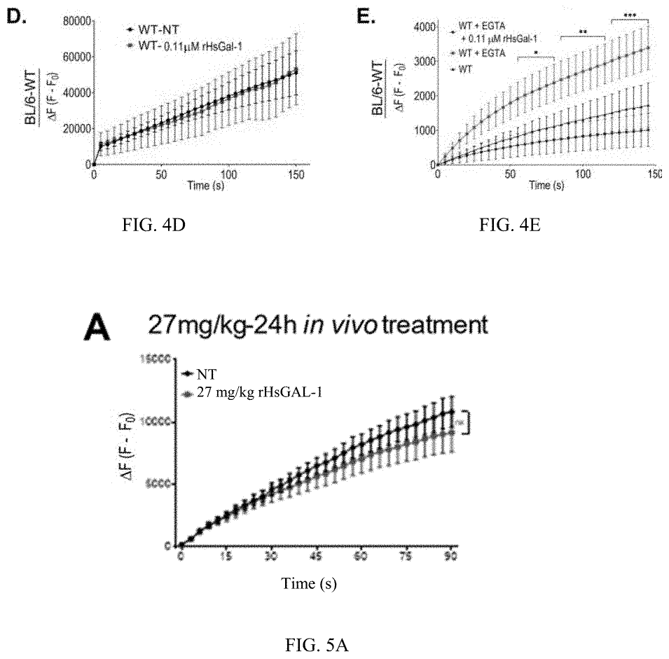

To verify in vitro myotube injury results, myofibers taken from Dysf^{-/-} and Bla/J mice were treated with 0.11 μM rHsGal-1 for 2 h prior to injury. Our results showed a 70% decrease in final fluorescent intensity from NT in the Dysf^{-/-} myofiber and a 57% decrease compared to NT in the myofiber from the Bla/J mice (FIG. 4A-4C). Injured mice fibers taken from C57BL/6 (WT) mice treated with or without rHsGal-1 showed no significant differences in membrane repair (FIG. 4D). Additionally, we used EGTA to inhibit the normal, calcium-dependent function of dysferlin in WT myofibers. When treated with EGTA, WT myofibers showed an increased dye entry of 50% compared to WT without EGTA. However, WT myofibers treated with EGTA plus 0.11 μM rHsGal-1 were not significantly different from WT myofibers untreated with EGTA or rHsGal-1 (FIG. 4E). These ex vivo results give further weight to in vitro myotube data.

rHsGal-1 Localizes at the Site of Injury and Sites of Cellular Fusion in Dysferlin-Deficient Myotubes

We next examined temporal-spatial localization of rHsGal-1 during laser injury and during myotube formation using AlexaFluor-647 conjugated rHsGal-1 (647rHsGal-1). 647rHsGal-1 localized on the membrane of myotubes after 10 min incubation. However, after a 48 h treatment there was minimal rHsGal-1 localized on the myotube membrane and instead formed puncta within the cytosol, further indicating the stability of the exogenous Gal-1 within these cells. After laser injury in the 48 hours 647rHsGal-1 treated myotubes, we observed 647rHsGal-1 concentrate at the site of injury. Confluent A/J^{-/-} myoblasts treated with 647rHsGal-1 in differentiation media for 10 minutes, 4 hours, 8 hours, 24 hours, and 48 hours were imaged to resolve differences in membrane versus nuclear localization. 647rHsGal-1 in confluent myoblasts treated for 10 minutes accumulated on the membrane and intramembrane space. By 4 hours of treatment, 647rHsGal-1 dispersed throughout the intracellular and extracellular space. Beginning at 4 hours and 8 hours, 647rHsGal-1 appears to coalesce in the shape of an extracellular lattice which expands in both 24 hours and 48-hour images. 48-hour post-treatment, we saw mature myotubes with intracellular rHsGal-1 and extracellular lattice structures of rHsGal-1 at sites of cellular fusion. Quantification of our results show after 4-hour treatment 647rHsGal-1 is predominately located inside myoblasts but by 8 hours and beyond most of the rHsGal-1 is found outside the cells. Additionally, we saw 647rHsGal-1 encapsulated in lipid layers, suggesting the formation of vesicles.

Current therapeutic options for LGMD2B are chiefly palliative in nature and do not present a significant quality of life benefit sought for by patients and their families Steroid treatment to reduce chronic inflammation is negatively correlated with patient muscle strength and poses significant negative side effects. Therefore, the need for developing an effective long-term treatment is imperative. Here, we demonstrated that the ability of rHsGal-1 to improve myogenic factors and membrane repair reflects its therapeutic potential to decrease disease pathologies in LGMD2B.

Dysferlin-deficient muscle cells show a marked decrease in myogenic potential. After treatment with rHsGal-1, the expression of myogenic transcription factors reveals that dysferlin-deficient cells are committing to a myogenic lineage and maturing faster than non-treated dysferlin-deficient cells. The removal of treatment after a 10 minutes rHsGal-1 followed by 72-hour differentiation was not sufficient to induce differences in myogenic, however, continuous 72 hours rHsGal-1 treatment coincided with increases in middle and late-stage markers. These results coupled with the formation of large multinucleated myotubes suggest that Gal-1 may help satellite cell commitment (FIGS. 2A and 2B). During in vivo muscle development, self-alignment of myotubes during myogenesis is crucial to form healthy myofibers. Self-alignment is increased due to Gal-1 treatment in myotubes, indicating that an in vivo Gal-1 treatment has the potential for similar results (FIG. 2F).

Ca²⁺ sensitive C2 domains of dysferlin aid in plasma membrane resealing, a necessary process in myogenesis and wound healing. In dysferlin-deficient myofibers and cells, this process is compromised, which leads to diminished reseal capacity after injury, perpetuating LGMD2B disease pathology. Kinetic laser injury results show that a 10-minute and 48-hour treatment improves membrane sealing; interestingly, the 10-minute treatment provided optimal membrane repair without upregulating myogenesis (FIG. 3B). However, this implies that rHsGal-1 induced improvement in myogenic potential alone cannot be responsible for the dramatic improvements in membrane sealing for this immediate result. Mechanical stabilization on the membrane due through the CRD of Gal-1 with known glycosylated membrane bound, EMC or yet to identified ligand could account for this action.

Differences in Ca²⁺ involvement, along with temporal-spatial localization, helps narrow down possible mechanistic pathways responsible for observed increases in repair in a LGMD2B system. Ex-vivo results suggest that rHsGal-1 treatment improves membrane repair capacity in two different dysferlin-deficient strains of mice. Moreover, rHsga1-1 will not alter normal membrane repair functionality at this dose and is independent of Ca²⁺ as we showed in A/J^{-/-} and A/J^{+/+} myotubes. One hypothesis that may offer explanation towards increase membrane repair capacity independent of Ca²⁺ is rHsGal-1 treatment upregulates crucial membrane repair proteins such as ANXA1 and ANXA6. The upregulation of ANXA1 and ANXA6 could also be attributed to differences in the rate of myogenesis since they are upregulated with differentiation.

These results indicate that the CRD of rHsGal-1 is an active structure required for therapeutic effect of Gal-1 in increasing membrane repair capacity (FIG. 3). We believe the CRD provides mechanical stabilization to the membrane, aiding other cellular repair machinery to effectively mitigate damage and enhance repair. Mechanical stabilization also explains calcium-independent action for membrane repair and the difference in membrane repair seen at 10 minutes versus 48 hours.

Cumulative results from this study provide evidence that rHsGal-1 may be a feasible protein therapeutic for LGMD2B by orchestrating a variety of changes that overcome intrinsic defects in myogenic functions. Increased connectivity observed in labeled rHsGal-1 may result in increased cellular signaling suggesting a potential mechanism for Gal-1 induced membrane repair that needs further investigation. Previous findings indicate localization of Gal-1 in the ECM. The appearance of increased deposition of labeled rHsGal-1 in the extracellular space herein indicates that Gal-1 may increase skeletal muscle integrity in animal models of dysferlinopathy. These cumulative results support the hypothesis that the CRD mechanistically binds glycosylated membrane associated proteins, providing stability and overcoming inherent membrane destabilization due to lack of dysferlin. Although questions still remain about the nature of rHsGal-1 therapeutic mechanisms and systematic effects in more complex models of LGMD2B, these results provide evidence that Gal-1 is a viable therapeutic candidate in muscle diseases.

### Example 2

Monomeric and dimeric forms of galectin-1 (mGal-1 and dGal-1) were successfully expressed from constructs received, purified and tested in membrane repair in A/J^{-/-} myotubes and BlaJ myofibers.

Both the oxidized and reduced versions of mGal-1 had very little effect on membrane resealing when cells were treated 10 min before wounding but did have intermediate beneficial effects on membrane resealing when administered to cells 48 hrs prior to wounding. This suggests that these variants may need to be internalized to have a benefit on membrane resealing.

rHsGal-1 was substantially more effective at improving membrane resealing than either monomeric form at both 10 minutes and 48 hours, suggesting that the fixed monomeric form is unable to achieve mechanistic stabilization. Our data shows that rHsGal-1 behaves similar to endogenous Gal-1 which changes between monomeric and dimeric forms based on concentration and cellular need.

Alkylated rHsGal-1 increases membrane repair levels comparable to the native reduced form of rHsGal-1. Alkylation of Galectin-1 prevents oxidation to keep an active CRD and increases storage life.

rHsGal-1 improves membrane repair capacity better than mGal-1 after a 10-minute treatment. Oxidation or reduction of mGal-1 doesn't improve its membrane repair ability. Reduced dGal-1 improves membrane repair, whereas oxidized dGal-1 does not.

### Example 3

In Vivo rHsGal-1 Treatment: Dose Response.

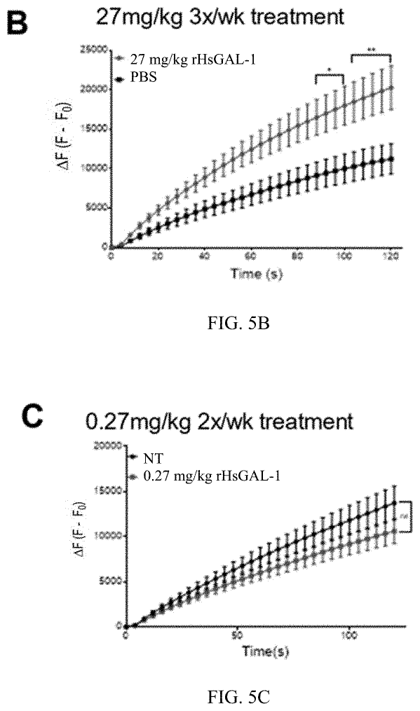

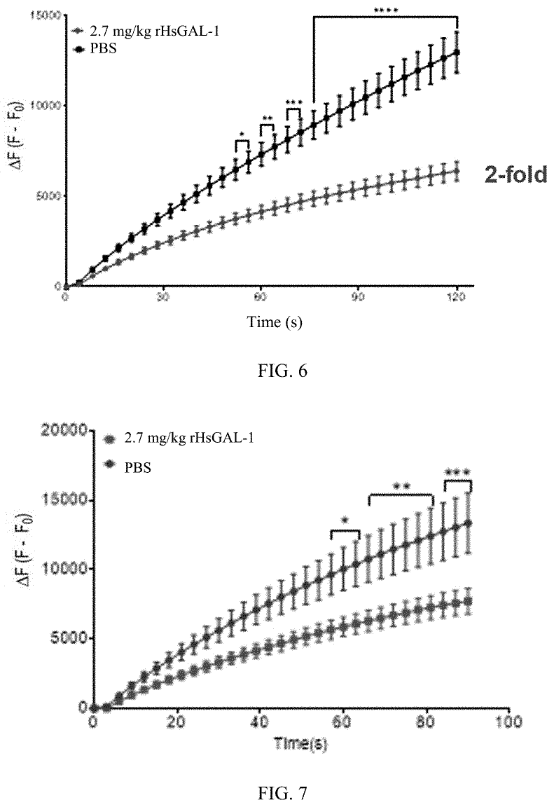

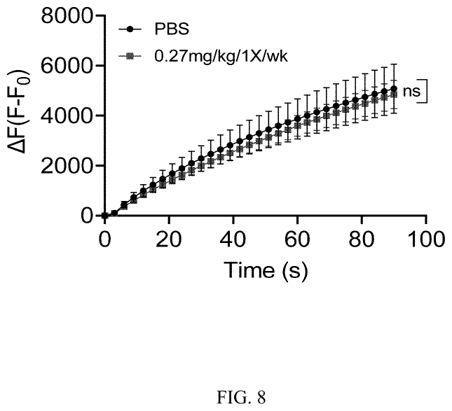

TABLE 1

| Length of Treatment | | Dose | | Treatment Regimen | Result |
|---|---|---|---|---|---|
| 24 | hours | 27 | mg/kg | once | FIG. 5A |
| 1 | week | 27 | mg/kg | 3x/week | FIG. 5B |
| 1 | week | 0.27 | mg/kg | 2x/week | FIG. 5C |
| 1 | week | 2.7 | mg/kg | 2x/week | FIG. 6 |
| 1 | month | 2.7 | mg/kg | 1x/week | FIG. 7 |
| 1 | month | 0.27 | mg/kg | 1x/week | FIG. 8 |

Initial dose finding experiments in mice demonstrate that 2.7 mg/kg dose (2×/wk) of rHsGal-1 induces a 2-fold improvement in membrane repair. The timing of the dose is important as dosing shortly before the animals were used for the experiment dramatically improved the benefits to membrane repair. Administration rHsGal-1 tends to promote further endogenous expression of the protein suggesting that repeated administration may lead to sustained elevated levels.

Dosing at 27 mg/kg 2×/wk only had minor benefits to membrane repair, suggesting this dose was inadequate to provide the full benefit. A very high dose treatment (27 mg/kg 3×/wk) made membrane repair worse in the animals.

Injection of 2.7 mg/kg 2×/wk for 1 week of rHsGal-1 into 36-42-week-old Bla/J mice. This treatment was an injection of 2.7 mg/kg at day 0 and 2 hours prior to culling mice, thus a 2-time treatment. Membrane repair of teased muscle fibers from the treated animals repaired significantly better than untreated animals (2.02 times better by the Van Ry lab's metric).

A study was done to determine if the time from the most recent injection makes a difference in the membrane repair assessment. Bla/J animals were treated with 2 injections of 2.7 mg/kg rHsGal-1 with the second injection occurring either 2 hours or 2 days prior to sacrifice for membrane repair assessment. In both cases the treated animals show better membrane repair than untreated controls and show no statistical different between treatments. However, the trend favors the 2 hours over the 2-day treatment suggesting there may be some benefit to the 2-hour treatment in membrane repair.

Injection of 2.7 mg/kg 1×per week for 1 month of rHsGal-1 into 43-50-week-old Bla/J mice. Membrane repair of teased muscle fibers from the treated animals repaired significantly better than untreated animals (1.6 times better by the Van Ry lab's metric).

1 month of treatment with 0.27 mg/kg 1×per week of rHsGal-1 was done and these mice showed no improvement in membrane repair when compared to PBS treated Bla/J animals. The results demonstrate that this dose is too low to show any benefit, but is also a nice negative control for the experiments above. No significant differences were shown between PBS and 0.27 mg/kg/1×/week 1-month treated myofibers. This treatment regimen also seemed to improve spontaneous rearing compared to untreated Bla/J as measured in CLAMS (activity) cages. This suggests the mice are doing better functionally.

### Statements

Statement 1: A method of treating limb-girdle muscular dystrophy type 2B in a patient, comprising administering a suitable amount of a galectin protein, a fragment thereof, or synthetic variant thereof.

Statement 2: The method of statement 1, wherein the suitable amount of the galectin protein or the fragment thereof is from about 0.2 mg/kg to about 20 mg/kg administered via intraperitoneal injection.

Statement 3: The method of statement 1, wherein the suitable amount of the galectin protein or the fragment thereof is from about 0.5 mg/kg to about 10 mg/kg administered via intraperitoneal injection.

Statement 4: The method of statement 1, wherein the suitable amount of the galectin protein or the fragment thereof is from about 1 mg/kg to about 5 mg/kg administered via intraperitoneal injection.

Statement 5: The method of statement 1, wherein the suitable amount of the galectin protein or the fragment thereof is from about 2 mg/kg to about 4 mg/kg administered via intraperitoneal injection.

Statement 6: The method of any one of statements 1-5, wherein the galectin protein is a recombinant galectin-1 protein.

Statement 7: The method of statement 6, wherein the recombinant galectin-1 protein is SEQ ID NO: 1.

Statement 8: The method of any one of statements 1-7, wherein the galectin protein or the fragment thereof is dosed daily or every other day.

Statement 9: The method of any one of statement 1-7, wherein the galectin protein or the fragment thereof is dosed weekly.

What is claimed is:

1. A method of treating limb-girdle muscular dystrophy type 2B in a patient, comprising administering a suitable amount of a galectin protein, a fragment thereof, or synthetic variant thereof.

2. The method of claim 1, wherein the suitable amount of the galectin protein or the fragment thereof is from about 0.2 mg/kg to about 20 mg/kg administered via intraperitoneal injection.

3. The method of claim 1, wherein the suitable amount of the galectin protein or the fragment thereof is from about 0.5 mg/kg to about 10 mg/kg administered via intraperitoneal injection.

4. The method of claim 1, wherein the suitable amount of the galectin protein or the fragment thereof is from about 1 mg/kg to about 5 mg/kg administered via intraperitoneal injection.

5. The method of claim 1, wherein the suitable amount of the galectin protein or the fragment thereof is from about 2 mg/kg to about 4 mg/kg administered via intraperitoneal injection.

6. The method of claim 1, wherein the suitable amount of the galectin protein or the fragment thereof is from about 0.01 mg/kg to about 5 mg/kg administered intravenously.

7. The method of claim 1, wherein the galectin protein is a recombinant galectin-1 protein.

8. The method of claim 7, wherein the recombinant galectin-1 protein is SEQ ID NO: 1, SEQ ID NO: 2, or SEQ ID NO: 3.

9. The method of claim 1, wherein the galectin protein or the fragment thereof is dosed daily or every other day.

10. The method of claim 1, wherein the galectin protein or the fragment thereof is dosed weekly.

11. The method of claim 1, further comprising decreasing muscle damage, increasing muscle repair, increasing muscle function, or any combination thereof.

12. A method of treating limb-girdle muscular dystrophy type 2B in a patient, comprising administering about 0.2 mg/kg to about 20 mg/kg of a galectin protein, a fragment thereof, or synthetic variant thereof, wherein the administration is via intraperitoneal injection.

13. The method of claim 12, wherein the suitable amount of the galectin protein or the fragment thereof is from about 0.5 mg/kg to about 10 mg/kg.

14. The method of claim 12, wherein the suitable amount of the galectin protein or the fragment thereof is from about 1 mg/kg to about 5 mg/kg.

15. The method of claim 12, wherein the suitable amount of the galectin protein or the fragment thereof is from about 2 mg/kg to about 4 mg/kg.

16. The method of claim 12, wherein the recombinant galectin-1 protein is SEQ ID NO: 1, SEQ ID NO: 2, or SEQ ID NO: 3.

17. The method of claim 12, wherein the recombinant galectin-1 protein is SEQ ID NO: 1.

18. The method of claim 12, wherein the recombinant galectin-1 protein is SEQ ID NO: 2.

19. The method of claim 12, wherein the recombinant galectin-1 protein is SEQ ID NO: 3.

20. The method of claim 12, further comprising decreasing muscle damage, increasing muscle repair, increasing muscle function, or any combination thereof.
